(19) United States
(12) Patent Application Publication
(10) Pub. No.: US 2024/0315684 A1
(43) Pub. Date: Sep. 26, 2024

(54) MEDICAL INSTRUMENT, SYSTEM, AND METHOD FOR SPRAYING LIQUIDS

(71) Applicant: Erbe Elektromedizin GmbH, Tuebingen (DE)

(72) Inventors: Frank STRAUB, Reutlingen (DE); Andreas FECH, Tuebingen (DE)

(21) Appl. No.: 18/608,085

(22) Filed: Mar. 18, 2024

(30) Foreign Application Priority Data
Mar. 23, 2023 (EP) 23163809.9

(51) Int. Cl. A61B 17/00; A61B 90/00

(52) U.S. Cl. CPC A61B 17/00491; A61B 2017/00495; A61B 2017/00522; A61B 2090/0816

(57) ABSTRACT

An instrument is used to spray a plurality of mutually reactive liquids by means of at least one swirling gas stream and to mix them outside the nozzle arrangement of the instrument. The nozzle arrangement of the instrument has a nozzle body with at least one liquid outlet, through which one or more liquids can be dispensed, and a gas outlet arrangement, through which the at least one swirling gas stream can be dispensed, the gas outlet arrangement enclosing the at least one liquid outlet.

### CROSS REFERENCE TO RELATED APPLICATION

This application claims the benefit of European Patent Application No. 23163809.9, filed Mar. 23, 2023, which is incorporated herein by reference in its entirety.

### TECHNICAL FIELD

The invention relates to a medical instrument, preferably for minimally invasive use with a nozzle arrangement arranged distally on the instrument, which is adapted to spray liquids by means of a swirling gas stream and to a system which, has this instrument. The invention also relates to a method for spraying medical liquids.

### BACKGROUND

During surgical interventions inside a patient's body, in particular in the abdominal region of the patient, pathological adhesions may form between the individual organs and/or the abdominal wall. These pathological adhesions are also known as adherences, which can sometimes lead to serious implications for patients after the procedure.

So-called adhesion barriers are used to prevent such adhesions as described for example in WO 2015/176 905 A1. These adhesion barriers are substances that can be applied to the affected tissue area in order to prevent other tissue layers from growing onto them. The adhesion barriers are advantageously applied by spraying. Preferably, these adhesion barriers are formed from two components that are only mixed together immediately before or during application.

Various systems for spraying several components for adhesion prevention are known from the prior art, which are also used in particular in the laparoscopic field.

WO 0 009 199 A1 describes a device, which is designed for spraying a two-component adhesion barrier. The device has separate spray nozzles for each of the two components. The components are sprayed out via a gas stream, whereby the gas stream exits through circumferential gaps surrounding the nozzles.

EP 3 145 417 A1 describes a second device for spraying at least two components using a pressurized gas inside the body of a patient. The device has a spray head in which the components emerge separately from one another and are mixed and sprayed by a gas stream that also emerges at the spray head.

Further spraying devices for several components are described in EP 0 951 311 B1, EP 0 302 411 A1, EP 2 739 401 A2, US 2006/189 944 A1 and EP 2 695 626 A1.

U.S. Pat. No. 5,368,563 A also describes an externally mixing nozzle for a physiological adhesive. In this nozzle the liquid components are swirled inside the nozzle so that the liquid components mix in the resulting spray cone outside the nozzle during spraying.

EP 2 907 582 B1 also describes an externally mixing nozzle in which at least two fluids with different volume flows and/or different viscosities are sprayed

In addition, JP 2011-194304 A describes a device for spraying two components forming an adhesive in which a rotating gas stream is used to spray the components. The outlets opening of the gas flow is located centrally between the two separate outlet openings for the components.

### SUMMARY

Based on this, it is one object of the invention to provide a medical instrument and a method for spraying liquid components, which are mixed outside the instrument in which, in particular, an improved mixing of the liquid components can be achieved.

This object is solved by the medical instruments, methods and systems disclosed herein.

The medical instrument according to the invention has a nozzle arrangement arranged distally on the instrument, which is adapted to spray liquids by means of a swirling gas stream. The nozzle arrangement has a nozzle body with a liquid outlet and a gas outlet arrangement. The instrument is arranged to dispense a liquid stream through the liquid outlet and a swirling gas stream through the gas outlet arrangement. The gas outlet arrangement is arranged in such way that it surrounds the liquid outlet, preferably completely, so that the swirling gas stream has as large of an overlap area as possible with the liquid stream.

The gas stream spreads from the gas outlet arrangement in a direction of propagation with a spray angle determined by the gas outlet arrangement. In addition to the flow component pointing in the direction of propagation, the gas stream is subjected to a vortex flow (hereinafter also referred to as swirl). When the gas stream catches the liquid droplets of the liquid stream in the overlap area these are entrained and mixed by the gas stream. Due to the vortex flow (the swirl) imparted to the gas stream additional shear forced act on the liquid droplets of the liquid stream causing the liquid droplets to be torn apart into finer liquid droplets in flight. As a result, a droplet spectrum with smaller droplet sizes can be achieved. The swirling gas stream can thus achieve improved mixing of the liquids outside the nozzle arrangement.

A special feature of the medical instrument according to the invention is that a swirling gas stream is generated by the nozzle arrangement, which is used for mixing and spraying the liquids outside the nozzle arrangement, whereby the liquids emerging at the nozzle arrangement are flowed around and/or through by the swirling gas stream, preferably in its entirety. The swirl imparted to the gas stream can lead to a larger spray angle of the gas/liquid mixture, whereby the application and distribution of the gas/liquid mixture can be facilitated and accelerated particularly in the case of large surfaces. By applying a swirl to the gas stream, a controlled, additional turbulence is imparted to the gas stream. The liquid droplets of the first liquid stream around, which the swirl gas stream flows are entrained and carried along a trajectory from the inside to the outside and/or from the outside to the inside, whereby the droplets are atomized as a result of the shear forced acting on them. The swirl imparted to the gas stream also ensures that the axial exit velocity of the gas/liquid mixture is reduced when the droplet size is small allowing the liquid to be applied evenly to the tissue.

The liquid to be sprayed is preferably composed of several mutually reactive components. The liquid to be sprayed can be a gel, for example, which is present in at least two components in its initial state. It is preferred that the gel is a hydrogel. A hydrogel is a water-retaining and at the same time water-insoluble polymer.

It is preferred that the hydrogel is based on polyurethane. The components of the liquid can be, for example, a prepolymer and a reaction partner required for crosslinking. These components react with each other upon mixing, wherein the reaction rate at which these components react with each other is comparatively high. For example, the components may have (fully) reacted with each other within 60 seconds or less, 50 seconds or less and 40 seconds or less or less at room or body temperature. The components are therefore preferably mixed outside the nozzle assembly to avoid clogging of the nozzle assembly after use.

In a first embodiment the nozzle arrangement has the first liquid outlet and a second liquid outlet through which a second liquid stream can be dispensed, wherein the second liquid outlet is arranged at a distance from the first liquid outlet. The second liquid outlet is preferably a separate liquid outlet. The first liquid outlet is preferably associated with a first liquid capillary through which a first feed flow of the first liquid is guided to the nozzle arrangement and the second liquid outlet is preferably associated with a second liquid capillary through which a second feed flow of the second liquid is guided to the nozzle arrangement. The two liquids to be mixed can thus be transported separately from a supply device to the nozzle arrangement of the instrument where they can be mixed and sprayed outside the nozzle arrangement by the swirling gas stream. By mixing the two liquids outside the nozzle arrangement clogging of the nozzle arrangement can be avoided. The distance between the first and second liquid outlets can be between 0.8 and 1.5 mm, preferably it is 1.4 mm, whereby good mixing of the liquids can be achieved and at the same time clogging of the nozzle arrangement can be prevented.

Preferably, the gas outlet arrangement has a first gas outlet opening, which is arranged concentrically to the first liquid outlet. The gas outlet opening can be an annular gap opening, for example. The annular gap opening can have a width of 0.08 to 0.15 mm, preferably a width of 0.1 mm. However, the gas outlet opening can also be divided into several annular gap segments. Alternatively, the gas outlet arrangement can have several gas outlet openings, which are arranged on a circle defined concentrically to the first liquid outlet.

In particular, the gas outlet arrangement has a second gas outlet opening, which is arranged concentrically to the second liquid outlet, wherein the first gas outlet opening and the second gas outlet opening are each adapted to dispense a swirling gas stream. The first liquid outlet and the second liquid outlet are thus each enclosed by a gas outlet opening. The swirling gas stream captures the liquids emerging from the liquid outlets and sprays them with the two resulting spray cones overlapping outside the nozzle arrangement. Improved mixing of the two liquid streams occurs in the overlapping area.

It is preferred that the swirling gas stream exiting through the first gas outlet opening and the second gas outlet opening are subjected to a swirl rotating in the same direction or rotating in opposite directions.

In a second embodiment the first liquid outlet and the second liquid outlet are arranged at a distance from one another in a flow body, which defines a central axis M.

In particular, the gas outlet arrangement has a gas outlet opening surrounding the flow body, which is preferably arranged concentrically to the central axis M of the flow body. The flow body preferably has a convex outer contour, whereby the gas stream follows the outer contour of the flow body s and entrainment of the two liquids can be improved. The surface of the flow body s is preferably designed with a hydrophobic coating. This can prevent droplets from adhering to the distal end of the instrument, which can otherwise lead to an accumulation of hardened (reacting with each other) liquid droplets, which in turn can block the gas outlet opening an/or the liquid outlets.

In the first and/or the second embodiment the swirling gas stream or the swirling gas streams preferably completely envelop the first liquid stream and the second liquid stream so that the liquid streams and the gas streams overlap as extensively as possible upstream of the nozzle arrangement. In this way the largest possible mixing zone for the liquids can be achieved.

Preferably, at least one gas supply channel opens into the nozzle body, whereby the gas outlet arrangement can be supplied with a gas supply stream via the gas supply channel. In particular, at least one swirl generating means is accommodated in the nozzle body, which is adapted to apply a predetermined swirl to the gas supply stream. The swirl generating means can, for example, be designed as a swirl body, which is arranged in such a way that the predetermined swirl is imparted to the gas supply stream. The swirl-generating means can also be designed as a connecting channel through which the gas supply stream is fed into the gas outlet opening in such way that the predetermined swirl is imparted to it. The gas swirl angle, which describes the angle of the swirl to the axis of rotation of the swirl can in particular be greater than 15°, preferably greater than 20° and particularly preferably greater than 25°.

In particular, the first liquid outlet and/or the second liquid outlet are arranged projecting distally from the instrument, which makes it more difficult for liquid droplets to adhere to the distal end of the instrument when the spraying process is interrupted. The gas streams flow over the liquid droplets so that liquid droplets adhering to the liquid outlets are entrained by the gas stream that has already flowed out even if the liquid stream and the gas stream are interrupted almost simultaneously.

Preferably, the instrument also has an elongated shaft in which the gas supply channel and the liquid capillary for each liquid outlet are arranged. The instrument can also have a proximally arranged interface device that can be connected to a supply device. The supply device may be arranged to supply the instrument with the liquids as well as the gas supply stream.

Preferably, the instrument further comprises a control unit adapted to interrupt the first liquid stream and, if present, the second liquid stream before the gas stream when the spraying process is terminated and the second gas stream, if present. The control unit can also be adapted to initiate the first gas stream before the first liquid stream when the spraying process starts and, if present, also to initiate the second gas stream before the second liquid stream. Both measures can help to avoid an accumulation of liquid droplets at the distal end of the instrument.

The method according to the invention for spraying liquids by means of an instrument, preferably of the above type comprises the following steps:
generating a first and/or a second swirling gas stream exiting through a gas outlet arrangement from the nozzle arrangement in a spraying direction;
generating a first liquid stream that exits through a liquid outlet into the first liquid gas stream; and
generating a second liquid stream, which exits through a second liquid outlet into the first gas stream or, if present, into a second swirling gas stream,
wherein the first swirling gas stream and/or the second swirling gas stream enveloping the first liquid stream and the second liquid stream, preferably completely.

In particular, when the spraying process ends, the first liquid stream and the second liquid stream are interrupted before the first gas stream and, if present, the second gas stream. Preferably, when starting the spraying process, the first gas stream and, if present, the second gas stream are initiated before the first liquid stream and the second liquid stream.

All the features and advantages described in relation to the instrument according to the invention are also applicable to the method according to the invention.

The system according to the invention has an instrument of the above type and a supply device for supplying the instrument with the required operating means. Operating means can be for example fluids, such as gases or liquids and/or an electrical voltage, in particular a high frequency alternating voltage.

### BRIEF DESCRIPTION OF THE DRAWINGS

Further details of advantageous further embodiments or details of the invention can be derived from the drawings, the description and the subclaims. It shows:

FIG. 1 a schematic view of an embodiment example of the instrument with a supply device;

FIG. 2 a cross-sectional view of the nozzle arrangement of the instrument according to a first embodiment example;

FIG. 3 a cross-sectional view of the nozzle arrangement of the instrument according to a second embodiment example;

FIG. 4 a longitudinal section of the nozzle arrangement of the instrument according to the first embodiment example;

FIG. 5 a longitudinal section of the nozzle arrangement of the instrument according to the second embodiment example;

FIG. 6 a distal top view of the nozzle arrangement of the instrument according to the third embodiment example;

FIG. 7 a longitudinal section of the nozzle arrangement of the instrument according to a third embodiment example;

FIG. 8 a longitudinal section of the modified nozzle arrangement of the instrument according to the third embodiment example; and

FIG. 9 a control scheme for a spraying process.

### DETAILED DESCRIPTION

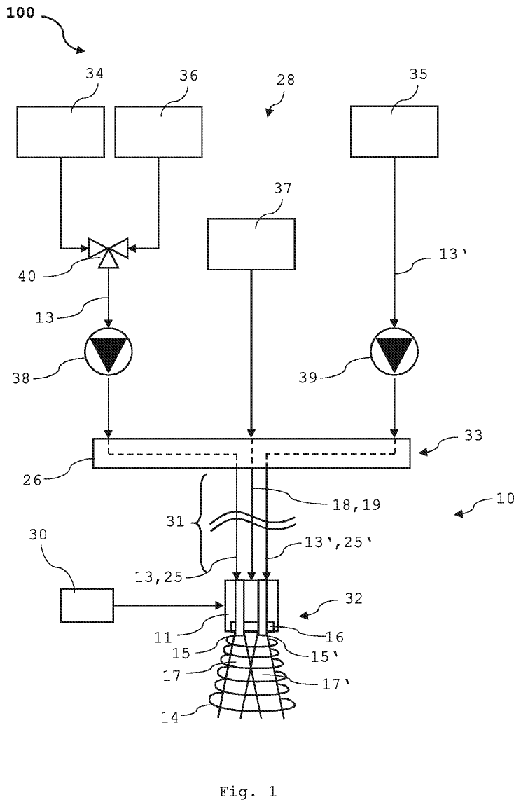

FIG. 1 shows a system 100 for supplying, mixing and spraying a plurality of liquid components. The system 100 has an instrument 10 and a supply device 28. The instrument 10 is preferably designed for minimally invasive use, in particular for laparoscopic use. The instrument 10 has a nozzle arrangement 11, which is arranged distally to the instrument 10. The nozzle arrangement 11 is designed to spray liquids 13 and 13′ by means of a first and/or a second swirling gas stream 14, 14′.

The nozzle arrangement 11 has a first liquid outlet 15 and a second liquid outlet 15′, through which a first liquid stream 17 of the first liquid 13 and a second liquid stream 17′ of the second liquid 13′ flow. The nozzle arrangement 11 further comprises a gas outlet assembly 16 adapted to dispense the first and/or second gas streams 14, 14′.

The instrument 10 has an elongated shaft 31 in which the liquids 13 and 13′ to be sprayed and the gas for generating the swirling gas stream are transported. At the proximal end 33 of the instrument 10 the instrument 10 has an interface device 26. The interface device 26 is adapted to be connected to a supply device 28. The supply device 28 provides the liquid 13 and the second liquid 13′.

The elongated shaft 31 of the instrument 10 has a liquid capillary 25, which is associated with the liquid 13 and a second separate liquid capillary 25′, which is associated with the second liquid 13′. The liquids 13 and 13′ are transported from the interface device 26 at the proximal end 33 of the instrument 10 to the nozzle arrangement 11 at the distal end 32 of the instrument 10 via the liquid capillaries 25 and 25′. A gas supply channel 18 is also disposed in the elongated shaft 31 of the instrument 10, the gas supply channel 18 transporting a gas supply stream 19 from the interface device 26 at the proximal end 33 to the nozzle assembly 11 at the distal end 32 of the instrument 10.

The supply device 28 has a first source 34 of a first component, a second source 35 of a second component, a third source 36 of a diluent and a fourth source 37 for a gas. The first component may, for example, be a prepolymer. The prepolymer in the first source 34 can have a relatively high viscosity, for example greater than 9 mPa·s, which is why diluent from the third source 36 can be added to it in the mixing device 40. The diluent can be water, for example. The first component mixed with the diluent forms the first liquid 13 which can be dispensed to the interface device 26 after premixing via a first dosing unit 38. The second component may be a catalyst. The second component forms the second liquid 13′, which can be dispensed from the supply device 28 to the interface device 26 via a separate metering unit 39. The second liquid can, for example, be a catalyst for the first liquid. The supply device 28 also has a fourth source 37 for the gas. The gas is preferably carbon dioxide or air or an inert gas, for example nitrogen or noble gases such as argon or the like. The gas from the fourth source 37 is supplied to the instrument 10 by the supply device 28 via the interface device 26. The sources 34, 35, 36, 37 may, for example, be reservoirs or tanks in which the components are stored. Alternatively, the sources 34, 35, 36, 37 can also be connections to a supply line of the component.

The instrument 10 also has a control unit 30, which is adapted to interrupt the liquid streams 17, 17′ when the spraying process ends before the gas streams 14, 14′, 14″. Likewise, the control unit 30 is adapted to activate the liquid stream 17 and 17′ at the start of the spraying process after the gas streams 14, 14′, 14″. In some embodiments, the control unit is embodied by a general-purpose circuit board that can accommodate various components of the internal electronic system of the instrument and further provide connections for other peripherals. More specifically, the circuit board provides the electronic connections by which the other components of the system can communicate electronically. Any suitable processor (inclusive of digital signal processors, microprocessors, supporting chipsets etc.) and computer readable, non-transitory memory elements etc. can be suitably coupled to the circuit board based on particular configuration needs, processing demands, computer designs, etc. Other components such as external storage, additional sensors, and controllers for peripheral devices may be attached to the circuit board as plug-in cards, via cables, or integrated into the board itself. In various embodiments, the functionality described herein may be implemented in emulation form as software or firmware running within one or more configurable (e.g. programmable) elements arranged in a structure that supports these functions. The software or firmware providing the emulation may be provided on non-transitory computer readable storage media comprising instructions to allow one or more processors to carry out those functionalities.

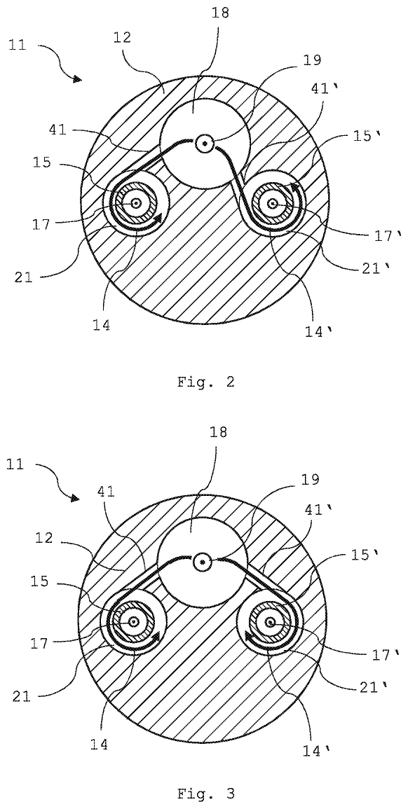

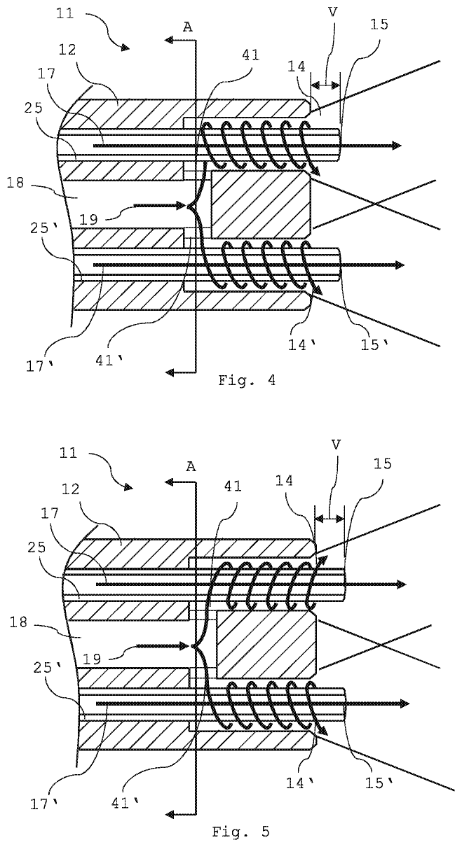

FIG. 2 shows a cross-section through the nozzle body 12 of the nozzle arrangement 11 of the instrument 10 according to a first embodiment example along the sectional plane A drawn in FIG. 4. A gas supply channel 18 is provided in the nozzle body 12 through which the gas supply stream 19 is guided. The gas supply stream 19 points out of the drawing plane.

A first liquid outlet 15 and a second liquid outlet 15′ are also arranged in the nozzle body 12. The first liquid stream 17 is dispensed through the first liquid outlet 15. The second liquid outlet 15′ is in turn provided for the second liquid stream 17′. The liquid outlets 15 and 15′ are each surrounded by a gas outlet opening 21 and 21′, which together form the gas outlet arrangement 16.

In addition, the nozzle body 12 houses a swirl generating means. In this embodiment example the swirl generating means is formed by connecting ducts 41, 41′ via which the gas outlet openings 21 and 21′ are fluidically connected to the gas supply duct 18. The connecting ducts 41 are arranged in such a way that the central gas supply stream 19 is divided into two partial gas streams. The partial gas streams are guided by the connecting ducts 41, 41′ into the gas outlet openings 21, 21′ in such a way that a first swirling gas stream 14 is formed in the first gas outlet opening 21 and a second swirling gas stream 14′ is formed in the second gas outlet opening 21′. The partial gas streams are introduced through the connecting ducts 41, 41′, preferably tangentially to the liquid outlets 15, 15′ into the gas outlet openings 21, 21′ of the gas outlet arrangement 16. The partial flows can flow into the gas outlet openings 21, 21′ at an inflow angle relative to a cross-sectional plane of the nozzle body 12. In this embodiment example the connecting ducts 41, 41′ are arranged in such a way that the first and second gas streams 14, 14′ flow in the same direction of rotation. Additionally, or alternatively, swirl bodies 20, 20′ can be arranged in the gas outlet openings 21, 21′, which provide the first gas stream 14 and the second gas stream 14′ with a swirl.

FIG. 3 shows a cross-section along the sectional plane A from FIG. 5 according to a second embodiment example. For the example shown in FIG. 3, the previously described applies accordingly with reference to the reference signs, although the connecting ducts 41 are arranged in such a way that the gas streams 14 and 14′ rotate in opposite directions. FIG. 3 shows a cross-section along the sectional plane A of FIG. 5.

FIG. 4 illustrates a longitudinal section of the nozzle arrangement 11 according to the first embodiment example. The nozzle body 12 accommodates the gas supply channel 18, through which the gas supply stream 19 is fed to the nozzle arrangement 11. The first liquid capillary 25 and the second liquid capillary 25′, through which the first liquid stream 17 and the second liquid stream 17′ are guided, are also arranged in the nozzle body 12. The first liquid capillary 25 ends in the first liquid outlet 15 and the second liquid capillary 25′ ends in the second liquid outlet 15′.

In one end section of the nozzle arrangement 11 a gas outlet opening 21 is arranged concentrically around the first liquid outlet 15. In this example, the first gas outlet opening 21 is designed as an annular gap. The central gas supply channel 18 is connected to the first gas outlet opening 21 via a connecting channel 41. In the end section of the nozzle arrangement 11, a second gas outlet opening 21′ is also arranged concentrically around the second liquid outlet 15′. The second gas outlet opening 21′ is also designed as an annular gap, wherein the second gas outlet opening 21 is connected to the gas supply channel 18 via a connecting channel 41′. The connecting ducts 41 are arranged in such a way that the gas supply stream 19 is divided into two partial flows with a counter-clockwise swirl. The first swirling gas stream 14 and the second swirling gas stream 14′ rotate in the same direction. The first liquid outlet 15 and the second liquid outlet 15′ protrude distally from the instrument 10 by the distance V.

FIG. 5 shows a longitudinal section of the second example of the nozzle arrangement 11. With regard to FIG. 5 the previously described also applies accordingly on the basis of the reference signs. FIG. 5 differs from FIG. 4 in that the connecting ducts 41 are arranged in such a way that the swirling gas stream 14 and the second swirling gas stream 14′ rotate in opposite directions.

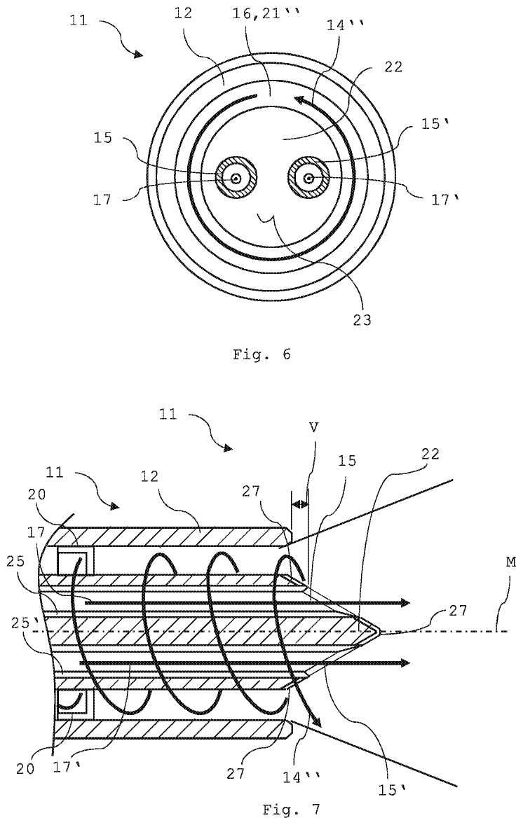

FIG. 6 shows a distal top view of the nozzle arrangement 11 according to the third embodiment example. In this embodiment example the liquid outlets 15 and 15′ are arranged in a common flow body 22. The flow body 22 is enclosed by the gas outlet arrangement 16. For this purpose, the gas outlet arrangement 16 has a gas outlet opening 21″ surrounding the flow body 22. This gas outlet opening 21″ is also designed as an annular gap. To generate the swirling gas stream 14″ surrounding the flow body 22, a swirler 20 is provided in the nozzle body 12, which imparts an additional angular momentum to the gas supply stream 19.

FIG. 7 shows an embodiment example of the nozzle arrangement, in which the two liquid outlets 15 and 15′ are arranged in the flow body 22. The flow body 22 is surrounded by the gas outlet opening 21″. At least one swirl body 20 is arranged in the gas outlet opening 21″, which imparts a rotational momentum to the gas supply stream 19. The outer contour of the flow body 22 is designed as a cone. It is also possible that the outer contour of the flow body 22 is designed as a truncated cone. The surface 23 of the flow body 22 has an additional coating 27. The coating can have hydrophobic properties for example.

FIG. 8 shows a second example of a nozzle arrangement in which the liquid outlets 15 and 15′ are provided in a flow body 22. The flow body 22 has a convex outer contour. In the example shown in FIG. 8, a coating is also provided on the surface 23 of the flow body 22, which can be hydrophobic, for example.

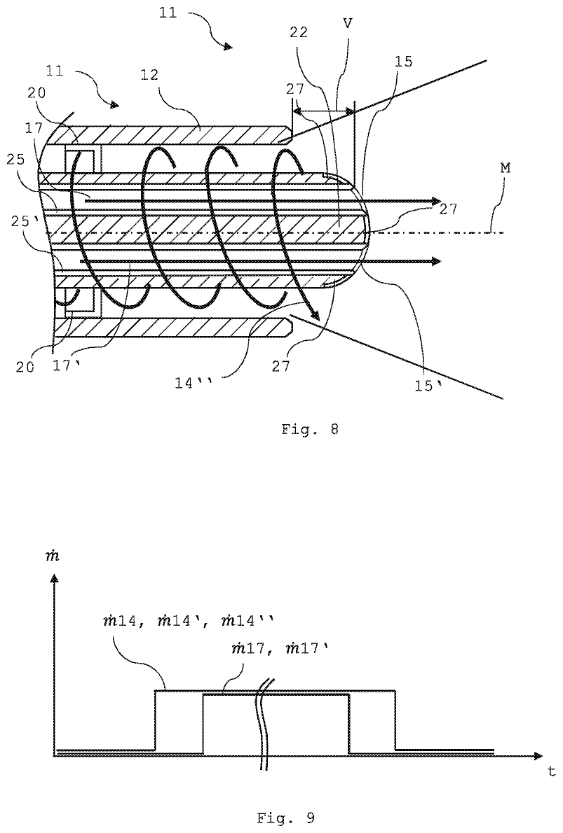

FIG. 9 shows the mass flows \dot{m} of the first gas stream 14 or second gas stream 14′ or third gas stream 14″ and the first liquid stream 17 or second liquid stream 17′ over the time t. The respective mass flows are labeled \dot{m}14, \dot{m}14′, \dot{m}14″ and \dot{m}17, \dot{m}17′. At the start of the spraying process, the mass flow \dot{m}14, \dot{m}14′, \dot{m}14″ of the gas stream 14 or 14′ or 14″ is introduced first, before the mass flows \dot{m}17, \dot{m}17′ of the liquid streams 17, 17′ are introduced. At the end of the spraying process, the mass flows \dot{m}17, \dot{m}17′ of the liquid streams 17, 17′ are correspondingly interrupted before the mass flows \dot{m}14, \dot{m}14′, \dot{m}14″ of the gas streams 14, 14′, 14″.

The instrument 10 according to the invention is used to spray a plurality of mutually reactive liquids 13, 13′ by means of at least one swirling gas stream 14, 14′, 14″ and to mix them outside the nozzle arrangement 11 of the instrument 10. The nozzle arrangement 11 of the instrument 10 has a nozzle body 12 with at least one liquid outlet 15, 15′ through which one or more liquids 13, 13′ can be dispensed and a gas outlet arrangement 16 through which the at least one swirling gas stream 14, 14′, 14″ can be dispensed, the gas outlet arrangement enclosing the at least one liquid outlet, preferably completely.

### LIST OF REFERENCE SIGNS

10 instrument
11 nozzle arrangement
12 nozzle body
13 first liquid (prepolymer)
13′ second liquid (catalyst)
14 first swirling gas stream
14′ second swirling gas stream
14″ swirling gas stream
15 first liquid outlet
15′ second liquid outlet
16 gas outlet arrangement
17 first liquid stream
17 second liquid stream
18 gas supply channel
19 gas supply stream
20 swirler
21 first gas outlet opening
21′ second gas outlet opening
21′ gas outlet opening
22 flow body
23 surface of the flow body
24 shaft of the instrument
25 first liquid capillary
25′ second liquid capillary
26 interface device
27 coating
28 supply device
29 first liquid supply flow
29′ second liquid supply flow
30 control unit
31 elongated shaft of the instrument
32 distal end of the instrument
33 proximal end of the instrument
34 first source of the first component
35 second source of the second component
36 third source of the diluent (water)
37 fourth source of the gas
38 first dosing unit (pump)
39 second dosing unit (pump)
40 mixing unit
41 connecting channels
A sectional plane
\dot{m} mass flows
t time
V distance

1. An instrument, comprising:
a nozzle arrangement arranged distally on the instrument and is adapted to spray liquids using at least one swirling gas stream;
wherein the nozzle arrangement comprises a nozzle body with a liquid outlet and a gas outlet arrangement; and
wherein the instrument is configured to dispense at least a first liquid stream through the liquid outlet and the at least one swirling gas stream through the gas outlet arrangement, wherein the gas outlet arrangement surrounds the liquid outlet.

2. The instrument according to claim 1, wherein the liquid outlet of the nozzle arrangement includes a first and a second liquid outlet wherein a second liquid stream is dispensable from the second liquid outlet and, the second liquid outlet is spaced apart from the first liquid outlet.

3. The instrument according to claim 2, wherein the gas outlet arrangement has a first gas outlet opening which is concentric with the first liquid outlet.

4. The instrument according to claim 3, wherein the gas outlet arrangement has a second gas outlet opening which is concentric with the second liquid outlet, wherein the first gas outlet opening and the second gas outlet opening are configured to emit first and second swirling gas streams, respectively.

5. The instrument according to claim 4, wherein the first swirling gas streams emerging from the first gas outlet opening and the second swirling gas streams emerging from the second gas outlet opening each have a swirl rotation, wherein the swirl rotations of the first and second swirling gas streams are in a same direction or in opposite directions.

6. The instrument according to claim 2, wherein the first liquid outlet and the second liquid outlet are arranged spaced apart from each other in a flow body, which defines a central axis.

7. The instrument according to claim 6, wherein the gas outlet arrangement has a gas outlet opening, which surrounds the flow body and is concentric with the central axis of the flow body.

8. The instrument according to claim 6, wherein the flow body has a convex or conical outer contour.

9. The instrument according to claim 4, wherein the first swirling gas stream and the second swirling gas stream envelope the first liquid stream and the second liquid stream, respectively.

10. The instrument according to claim 1, further comprising a gas supply channel, which opens into the nozzle body and via which the gas outlet arrangement is supplied with a gas supply stream; and
at least one swirl generating means which is adapted to apply a predetermined swirl to the gas supply stream.

11. The instrument according to claim 2, wherein the first liquid outlet and/or the second liquid outlet project distally from the nozzle body.

12. The instrument according to claim 10, further comprising an elongated shaft in which the gas supply channel and a liquid capillary for each liquid outlet extend separately from each other; and
a proximally arranged interface configured to be connected to a gas and liquid supply which is configured to supply the instrument with the liquids and the gas supply stream.

13. The instrument according to claim 4, further comprising a control unit configured to interrupt the first liquid stream and the second liquid stream when a spraying process is terminated before the first gas stream and the second gas stream are terminated.

14. A method of spraying liquids with the instrument of claim 2 comprising:
generating the at least one swirling gas stream exiting through the gas outlet arrangement from the nozzle arrangement in a spraying direction;
generating the first liquid stream, which exits through the first liquid outlet; and
generating the second liquid stream, which exits through the second liquid outlet,
wherein the at least one swirling gas stream envelops at least the first liquid stream.

15. A system comprising the instrument according to claim 1, further comprising a supply for supplying the instrument with required operating means.

16. The method of claim 14, wherein the at least one swirling gas stream envelopes both the first and second liquid streams.

17. The instrument according to claim 7, wherein the gas outlet opening is configured to emit a swirling gas stream surrounding the flow body and the swirling gas stream envelopes the first liquid stream and the second liquid stream.
